(19) United States
(12) Patent Application Publication
(10) Pub. No.: US 2005/0010297 A1
(43) Pub. Date: Jan. 13, 2005

(54) Balloon technologies for tissue repair

(75) Inventors: John A. Watson, Dulliken (CH); Didier Cowling, Langnau (CH); Jason C. Schense, Zurich (CH); Dominik Ellenrieder, St. Sulpice (CH)

Correspondence Address: PATREA L. PABST;PABST PATENT GROUP LLP, 400 COLONY SQUARE, SUITE 1200, ATLANTA, GA, 30361, US

(73) Assignee: Kuros Biosurgery AG

(21) Appl. No.: 10/841,663

(22) Filed: May 7, 2004

Related U.S. Application Data
(60) Provisional application No. 60/469,354, filed on May 8, 2003.

(51) Int. Cl. A61M 29/00; A61M 31/00

(52) U.S. Cl. 623/17.12

(57) ABSTRACT

A medical device containing an inflatable balloon structure for use in minimally invasive surgery and minimally invasive diagnostic and therapeutic procedures are described herein. The device is delivered by a catheter and expanded using gases, liquids or liquids that solidify in situ. The inflatable balloon may be constructed from a wide variety of materials and may be reinforced by supporting structures, when necessary. The device may form an endoprosthesis in a patient. In the preferred embodiment, the device is used in spinal fusion. Optionally, the device may also be used in combination with bone graft materials and bioactive factors.

### CROSS REFERENCE TO RELATED APPLICATION

This application claims priority to U.S. Ser. No. 60/469,354, entitled “Balloon Technologies for Tissue Repair” to John Watson et al., filed May 8, 2003.

### BACKGROUND OF THE INVENTION

Developed in the late 1970s, high-pressure medical balloons have traditionally been used in angioplasty, a procedure that opens a blood vessel clogged by a build-up of fatty plaque. Recent advances in the design and fabrication of high-pressure medical balloons have enhanced their performance capabilities and broadened the use of medical balloons into new applications in the medical device industry.

Today's medical balloons, with thinner walls, higher strength, and smaller profiles, are well suited for use in a broad range of minimally invasive procedures. They can be produced in a variety of lengths, diameters, and shapes, including complex custom shapes for specific applications, and supplied with specialty coatings for added performance. Balloon coatings include formulations designed to modify lubricity (both hydrophilic and hydrophobic coatings), abrasion and puncture resistance, conductivity, thrombogenicity, drug release, among other characteristics. Currently, most medical balloons are made from either poly(ethylene terephthalate) (PET) or nylon. PET offers advantages in tensile strength and maximum pressure rating, whereas nylon is softer. Innovations in balloon design and technology have provided increased flexibility to product designers, making the development of new and improved devices possible. As a result, balloons are employed in a growing number of diagnostic and therapeutic procedures.

The small size of a deflated balloon makes its delivery via minimally invasive surgical techniques possible, thus limiting damage to the surrounding tissue. Balloons inflated with air or gases are often used to create/remove blockages, relocate damaged tissue or position medical devices such as stents.

Techniques presently used for spinal fusion require very invasive measures which can prolong recovery times and reduce the success rate. A fusion is a bridge of solid bone that is created by surgery and links the bones together to maintain alignment and provide stability and strength. Approximately 258,000 spinal fusions were performed in 1999. About 119,000 procedures involved the upper (cervical) spine. About 139,000 involved the lower (lumbar) spine. There are many reasons a surgeon may consider fusing vertebrae. These include treatment of fractured vertebrae, correction of deformity, elimination of pain, disc degeneration and instability. It is believed that pain originates in levels of the spine where the bones are slipped or the discs or joints are damaged and produce pain. This may be due to irritated nerve endings around the disc, bone or joints themselves or due to actual entrapment of the spinal nerves in that region. By eliminating motion across the damaged level, pain can be reduced. A solid bridge of bone eliminates motion that normally would take place at the disc space and in the joints of the spine.

Therefore it is an object of the invention to provide less invasive techniques for diagnostic and therapeutic procedures.

It is a further object of the invention to provide a device which can be used in less invasive diagnostic and therapeutic procedures.

### BRIEF SUMMARY OF THE INVENTION

A medical device containing an inflatable balloon structure for use in minimally invasive surgery and minimally invasive diagnostic and therapeutic procedures are described herein. The device is delivered by a catheter and expanded using gases, liquids or liquids that solidify in situ. The inflatable balloon may be constructed from a wide variety of materials and may be reinforced by supporting structures, when necessary. The device may form an endoprosthesis in a patient. In the preferred embodiment, the device is used in spinal fusion. Optionally, the device may also be used in combination with bone graft materials and bioactive factors.

### BRIEF DESCRIPTION OF THE DRAWINGS

FIG. 1 is a cross-section through the device and delivery catheter.

FIG. 2 is a vertical section through a portion of human spine into which the device is placed along with a bone graft substitute material.

FIG. 3 is a vertical section through a portion of human spine into which the device is placed.

### DETAILED DESCRIPTION OF THE INVENTION

I. Device

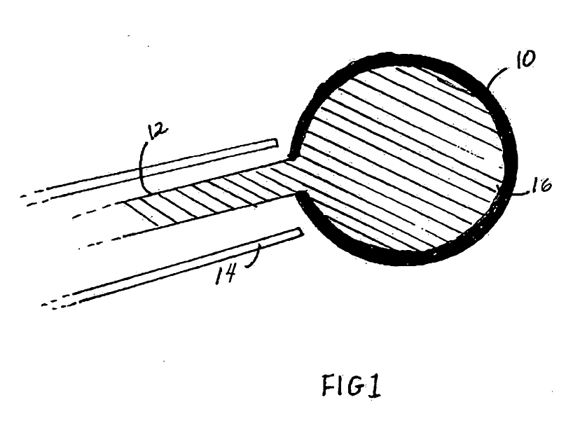

Devices containing an inflatable balloon or chamber may be used in therapeutic or diagnostic techniques. As depicted in FIG. 1, the medical device contains a collapsed balloon or chamber (10) that is connected to a catheter. FIG. 1 is a cross-section through the device and delivery catheter. The device includes a balloon body (10) which is formed of a hollow, inflatable, flexible material, such as PET or Kevlar. The device has one or more hollow tubes (12), together with a catheter (14) which communicate with and extend away from the balloon body (10), respectively, to a source of liquid or gas under pressure (not shown in FIG. 1). The liquid can be any sterile biocompatible solution (16). After the balloon has been inserted into the site in need of treatment in a collapsed condition or a situation where stabilisation is required, the liquid or gas inflates the balloon (10).

A. Inflatable Balloon or Chamber

After inserting the device into a patient, the balloon or chamber expands such that the final volume is significantly larger than the original volume of the chamber when it is placed into the patient. The chamber is expandable through many different methods. These methods include, but are not limited to, using spring driven methods (such as employed in stents), flexible materials in the wall of the chamber that allow the chamber to expand under pressure (similar to a balloon) and other options. The end result is an open chamber that is connected to a catheter. These chambers are standard chambers or high pressure chambers, which are capable of withstanding both large external and/or internal pressures without loss of integrity.

i. Shape

The shape and size of the expandable chamber may be varied depending on the application site. The shape can be a cylindrical shape, a sphere or many other options. As an example, if there is application in the spine, an expanded cylindrical chamber that is the approximately the size of a human vertebral disc that can support load my be required. However, if it is instead used in another location, a smaller or larger chamber may be required.

ii. Material

Another significant variable in these devices is the material that the chamber is made from. The material can be a simple single material design or a composite. The materials can be either non-degradable or biodegradable. The non-degradable materials are used when the chamber is expected to remain in the body of the patient for approximately the lifetime of the patient. The biodegradable materials are selected to degrade when the chamber has finished serving its function. Examples for possible choices of non-degradable material include rubbers, polymers such as polyethylene or polystyrene, Kevlar or many others. Biodegradable materials based on lactic and glycolic acid and on other materials, including poly(dioxanone), poly(trimethylene carbonate) copolymers, and poly (ε-caprolactone) homopolymers and copolymers, have been accepted for use in medical devices and are potential materials for the formation of the chamber. In addition to these approved materials, a great deal of research continues on polyanhydrides, polyorthoesters, polyphosphazenes, and other biodegradable polymers which may also be suitable. Since the device may be left in situ in the patient for a long period of time, both the mechanical performance of the balloon material over long time periods as well as the immunogenic properties of the material are important.

Optionally, the material includes a reinforcement. This reinforcement can be due to metal present in the walls of the chamber, outside the walls of the chamber, and in various configurations of metal. These include solid metal surfaces that are capable of providing support, metal meshes that share in carrying load and many other designs.

B. Gases or Liquids Inside the Balloon

The composition and character of the in situ polymerizing material that is used to fill the chamber can be selected to tailor the characteristics of the filled, polymerized chamber to the indication to be treated. The chamber can be filled with a non-degradable, highly crosslinkable material that results in a block. Some examples include rubbers, bone cements that are comprised of polymethylmethacrylate (PMMA) and other cement-like materials. Another type of material which can be used to fill the chamber an in situ crosslinkable polymer, whose liquid precursors are an electrophile triacrylate and a four nucleophile thiol group siloxane compound. Polymerization occurs due to mixing of these precursors together with a reaction starter, allowing the formation of covalent bonds between the electrophilic and nucleophilic groups following a Michael-type addition reaction. The solidification time is about fifteen minutes. However, the polymerization process, and therefore the increase in mechanical properties, may continue for a few days. The polymerization process results in the formation of a material with a compressive ultimate strength typically ranging from 5 to 25 MPa and a Young modulus, typically ranging from 50 to 150 MPa.

If the balloon is filled with either a liquid or a liquid that becomes solid in situ it may provide structural, load-bearing support for hard tissue. In this case, the balloon must be able to withstand high pressures. These types of materials are particularly suitable for applications where support or load bearing is required, such as for the repair of bone defects.

In contrast, in other indications the material filling the chamber should be a softer material. The softer material may be a softer rubber or a synthetic or natural material with a low crosslink density. These materials may be useful in indications where due to the lower pressures in the environment, a softer material is better suited.

Optionally, additional materials are added to the polymer precursors to produce a material with good radiopacity and/or thixotropic properties. For example, barium sulphate and silica particles may be included in the two precursors to confer good radiopacity and thixotropic properties, respectively, which are required when the material, still liquid, has to be injected in the human body under X-ray imaging.

B. Catheter

The chamber is pushed out of the catheter and inflates upon delivery of the gas or liquid into the chamber. In one embodiment, the catheter allows a liquid material to pass through it and into the chamber.

C. Bone Graft Material

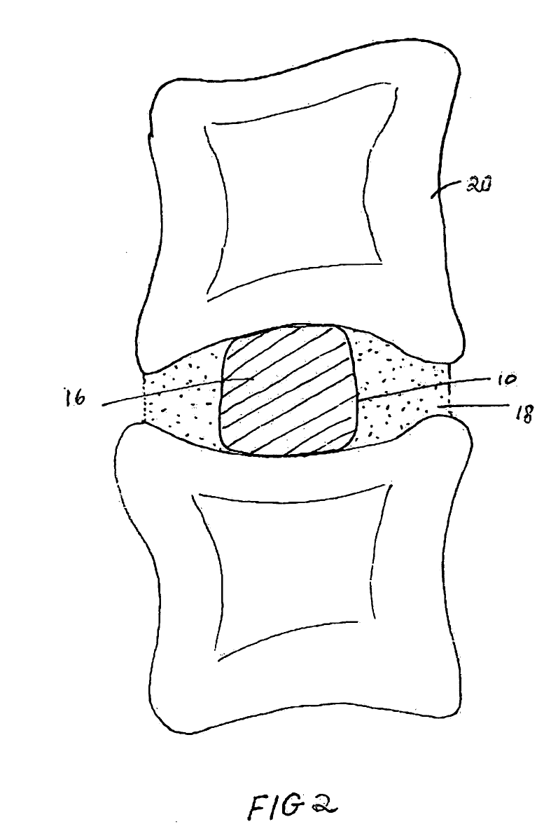

Stability is provided by a balloon filled under high pressure with a liquid orthopedic material that solidifies in situ to restore the height of the disc and provide support to the spinal column. As depicted in FIG. 2, the device may be designed such that after expansion, the expanded chamber is smaller than the entire disc space, but large enough to provide the required support after surgery. The remaining space between the device the annulus of the disc is filled with a bone graft substitute material (18). The balloon is placed centrally in the vertebrae (20). After the balloon has inflated and the filler has solidified, bone graft material (18) is placed around the device (10). This material may be a natural material such as fibrin, collagen or synthetic material, these will contain bone chips or bioactive factors, introduction of which will lead to spinal fusion.

The bone graft substitute material may be a variety of different materials including, but not limited to, autologous bone, granules of tri-calcium phosphate, hydroxyapatite or mixtures thereof, autologous blood clots, matrices with growth factors, including BMP-2 or OP-1 in collagen. In a preferred embodiment, the bone graft substitute material is a fibrin gel, optionally containing growth factors or peptides (whole or fragments thereof), such as parathyroid hormone, covalently bound to the matrices as described WO 01/83522 to Jeffrey Hubbell et al.

B. Methods of Using the Device

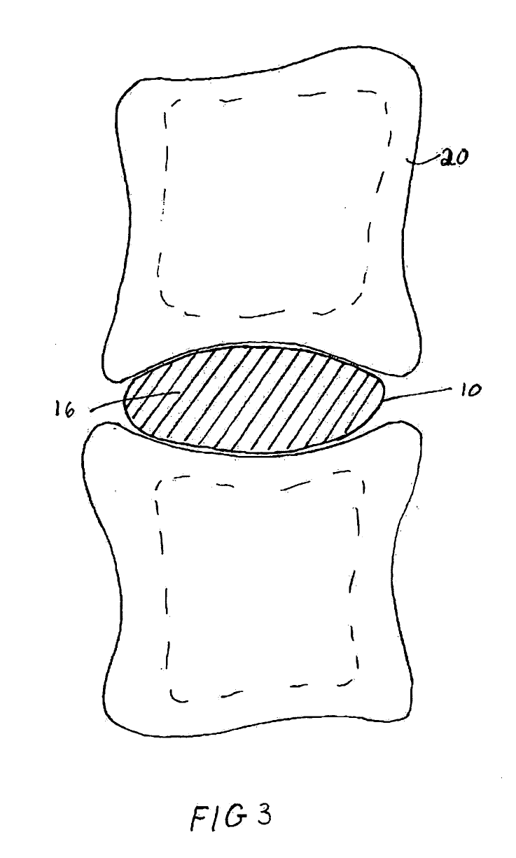

One primary indication for these devices will be in the spinal column. Presently, all devices applied to the column to assist in support of the column to repair a degenerated site are applied through very invasive techniques. In this embodiment of the invention, a reinforced device as described can be applied between two vertebrae in the area of the spinal disc, expanded to fill the space and then filled with a strong in situ polymerizing material. This will be done alone or in combination with other structural support such as pedicle screws. A picture of this is shown in FIG. 3.

As depicted in FIG. 3, balloon (10) is initially deflated and, after the cavity to be filled with the balloon has been prepared to receive the balloon, the deflated balloon is forced through the catheter (14). The balloon (10) is oriented preferably in a manner that allows it to exert maximum pressure on the surrounding vertebrae (20). Such pressure will provide stability to the spine.

In a related indication, these devices may also be employed as a substitute for the standard cage in spinal fusion indications. The use of the device in such an indication is depicted in FIG. 2. Here the device would be designed such that after expansion, the chamber (10) is smaller than the entire disc space, but large enough to provide the required support after surgery. The remaining space between the device the annulus of the disc is then filled with a bone graft substitute material (18).

Although spinal fusion is the preferred indication the device may be applied to any damaged tissue in order to relocate it and provide support, for example in the vertebrae or cancellous bone in the femur. This technology can also be used for creating or clearing blockages in blood vessels an other ducts within the body. The use of the device to deliver materials and actives to specific sites in the body, for example, radioactive materials in cancer therapy is another embodiment. The balloons described herein can also be used to position diagnostic devices inside vessels or body cavities.

Those skilled in the art will recognize, or be able to ascertain using no more than routine experimentation, many equivalents to the specific embodiments of the invention described herein. Such equivalents are intended to be encompassed by the following claims.

1. A medical device comprising an inflatable balloon, wherein the balloon is inflated by an in situ polymerizable material.

2. The medical device of claim 1, wherein the balloon is formed of a biodegradable material.

3. The medical device of claim 1, wherein the balloon is formed of a nondegradable material.

4. The medical device of claim 1, wherein the balloon further comprises reinforcing materials.

5. The medical device of claim 4, wherein the reinforcing materials are selected from the group consisting of solid metal surfaces and metal meshes.

6. The medical device of claim 1, wherein the balloon comprises pores.

7. The medical device of claim 1, wherein the balloon is suitable for orthopedic applications.

8. A method to facilitate minimal invasive surgery in orthopedic applications comprising
(a) inserting a medical device comprising an inflatable balloon in a non-inflated state at a site in need thereof, and
(b) inflating the balloon to a predetermined volume by polymerizing in situ a synthetic polymerizable material.

9. The method of claim 8, wherein the material comprises one or more liquids.

10. The method of claim 9, wherein the liquids are selected from the group consisting of polymethylmethacrylate and electrophiles and nucleophiles that undergo Michael-type addition reactions.

11. The method of claim 8, wherein the one or more liquids further comprise barium sulfate or silica particles.

12. The method of claim 8, further comprising (c) inserting a bone graft substitute material in the area surrounding the inflated balloon.

13. The method of claim 12, wherein the bone graft substitute material is selected from the group consisting of autologous bone, granules of tri-calcium phosphate, hydroxyapatite, autologous blood clots, collagen, and fibrin.

14. The method of claim 13, wherein the bone graft substitute material further comprises growth factors and peptides.
